(19) United States
(12) Patent Application Publication
(10) Pub. No.: US 2011/0046721 A1
(43) Pub. Date: Feb. 24, 2011

(54) Biodegradable Metal-Polymer Composite Constructs For Implantable Medical Devices

(75) Inventor: James H. Arps, Chanhassen, MN (US)

Correspondence Address: Pauly, Devries Smith & Deffner, L.L.C., Plaza VII, 45 South Seventh Street, Suite 3000, Minneapolis, MN, 55402-1630, US

(73) Assignee: SURMODICS, INC., Eden Prairie, MN (US)

(21) Appl. No.: 12/853,794

(22) Filed: Aug. 10, 2010

Related U.S. Application Data
(60) Provisional application No. 61/232,653, filed on Aug. 10, 2009.

(51) Int. Cl. A61F 2/82; A61F 2/00

(52) U.S. Cl. 623/1.38; 523/113; 424/426

(57) ABSTRACT

Embodiments of the invention include biodegradable composites and medical devices including the same. In an embodiment the invention includes a biodegradable implantable medical device. The implantable medical device can include a biodegradable composite member including a polymeric matrix and a reinforcing metal disposed within the polymeric matrix. The biodegradable composite member can be configured to erode in vivo. In an embodiment the invention includes a method of making a biodegradable implantable device including contacting a polymer mixture with a reinforcing metal, the polymer mixture comprising a polymer that degrades under in vivo conditions and the reinforcing metal comprising a metal that produces substantially non-toxic erosion products. Other embodiments are included herein.

This application claims the benefit of U.S. Provisional Application No. 61/232,653, filed Aug. 10, 2009, the contents of which are herein incorporated by reference.

### FIELD OF THE INVENTION

The present invention relates to medical devices. More specifically, the present invention relates to biodegradable composites and medical devices including the same.

### BACKGROUND OF THE INVENTION

Implantable medical devices are widely used as a part of modern medical care. Implantable medical devices can be used to provide mechanical support, provide fixation, deliver therapy, monitor physiological conditions, and replicate functions of bodily components, amongst other things. Implantable medical devices can include devices related to orthopedics, cardiology, neurology, urology, general surgery, plastic surgery, and many other medical applications.

Some implantable medical devices are designed to be transitorily implanted and then later removed. Other implantable medical device are designed to be implanted for an extended period of time and removed only at long intervals or if otherwise necessary. Still other medical devices are designed to be implanted permanently.

The ease of removing a medical device depends in significant part on the length of time that the device has been implanted. The immune reaction to a foreign body generally results in the formation of fibrous scar tissue around implants which can make later removal extremely difficult and potentially harmful to the patient.

### SUMMARY OF THE INVENTION

Embodiments of the invention include biodegradable composites and medical devices including the same. In an embodiment the invention includes a biodegradable implantable medical device. The implantable medical device can include a biodegradable composite member including a polymeric matrix and a reinforcing metal disposed within the polymeric matrix. The biodegradable composite member can be configured to erode in vivo.

In an embodiment the invention includes a method of making a biodegradable implantable device including contacting a polymer mixture with a reinforcing metal, the polymer mixture comprising a polymer that degrades under in vivo conditions and the reinforcing metal comprising a metal that produces substantially non-toxic erosion products.

The above summary of the present invention is not intended to describe each discussed embodiment of the present invention. This is the purpose of the figures and the detailed description that follows.

### BRIEF DESCRIPTION OF THE FIGURES

The invention may be more completely understood in connection with the following drawings, in which:

FIG. 1 is a schematic cross-sectional view of a composite in accordance with various embodiments.

FIG. 2 is a schematic cross-sectional view of a composite in accordance with various embodiments.

FIG. 3A is a schematic cross-sectional view of a composite in accordance with various embodiments.

FIG. 3B is a schematic cross-sectional view of a composite in accordance with various embodiments.

FIG. 4 is a schematic view of an implantable medical device in accordance with an embodiment.

FIG. 5 is a schematic cross-sectional view of an implantable medical device as taken along line 5-5′ of FIG. 4.

FIG. 6 is an enlarged view of a portion of FIG. 5.

FIG. 7 is schematic view of an implantable medical device in accordance with an embodiment.

FIG. 8 is a schematic cross-sectional view of an implantable medical device as taken along line 8-8′ of FIG. 7.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

As described above, the immune reaction to a foreign body generally results in the formation of fibrous scar tissue around implants which can make later removal extremely difficult and potentially harmful to the patient. However, that issue can be obviated if the device is biodegradable.

Embodiments herein include biodegradable composites and implantable medical devices including the same. Biodegradable composites including a degradable polymeric matrix and a reinforcing metal can have structural properties that exceed those of otherwise similar structures including only the degradable polymeric matrix yet can predictably degrade over a period of time. As such, biodegradable composites can allow for the implantation of a structurally robust device while eliminating the need for subsequent device removal.

The term “degradable polymer” as used herein shall refer to those natural or synthetic polymers that break down under physiological conditions into substantially non-toxic constituent components over a period of time. By way of example, many degradable polymers include hydrolytically unstable linkages in the polymeric backbone. The cleavage of these unstable linkages leads to degradation of the polymer.

The term “degradable metal” as used herein shall refer to metals that break down under physiological conditions into substantially non-toxic degradation products over time.

The term “biodegradable” as used herein with reference to implantable medical devices and composites shall refer to implantable medical devices and composites that substantially degrade over time after implantation in the body. The terms “bioerodible”, “biosorbable”, and “bioresorbable shall be subsumed within the term “biodegradable” as used herein.

Referring now to FIG. 1, a schematic cross-sectional view of a biodegradable composite 100 is shown in accordance with at least one embodiment. The biodegradable composite 100 can be used to form an implantable medical device and/or a member thereof. The biodegradable composite 100 includes a polymeric matrix 102 and a reinforcing metal 104. The polymeric matrix 102 can include a degradable polymer. Exemplary degradable polymers are described in greater detail below. The reinforcing metal 104 can include a metal, in elemental form, as an alloy, or as a metal oxide, that is configured to degrade in vivo. Exemplary reinforcing metals are described in greater detail below.

The biodegradable composite can be configured to degrade over a period of time. By way of example, in some embodiments the biodegradable composite can be configured to degrade in vivo at least about 90% in mass over a period of time no longer than 2 years. In some embodiments the biodegradable composite can be configured to degrade in vivo at least about 90% in mass over a period of time no longer than 12 months. In some embodiments the biodegradable composite can be configured to degrade in vivo at least about 90% in mass over a period of time no longer than 9 months. In some embodiments the biodegradable composite can be configured to degrade in vivo at least about 90% in mass over a period of time no longer than 6 months. In some embodiments the biodegradable composite can be configured to degrade in vivo at least about 90% in mass over a period of time no longer than 2 months.

The biodegradable composite, including a polymeric matrix and reinforcing metal, can exhibit enhanced structural properties in comparison with an otherwise identical member including only the polymeric matrix. In some embodiments the biodegradable composite can exhibit stiffness that is greater than an otherwise identical structure including only the polymeric matrix. In some embodiments the biodegradable composite can exhibit fracture resistance that is greater than an otherwise identical structure including only the polymeric matrix. In some embodiments the biodegradable composite can exhibit tensile strength (such as yield strength) that is greater than an otherwise identical structure including only the polymeric matrix. In some embodiments the biodegradable composite can exhibit a Young's modulus that is greater than an otherwise identical structure including only the polymeric matrix.

The biodegradable composite can be formed in various ways. In some embodiments, the polymeric matrix can be injection molded into a mold including the reinforcing metal. In some embodiments, the biodegradable composite can be deposited from a liquid composition including a polymer to form the matrix and the reinforcing metal. In some embodiments the polymers of the polymeric matrix can be mixed with the reinforcing metal and the resulting mixture can be cast into a desirable shape. In some embodiments the polymer can be infused into a woven or porous metal pre-impregnated substrate.

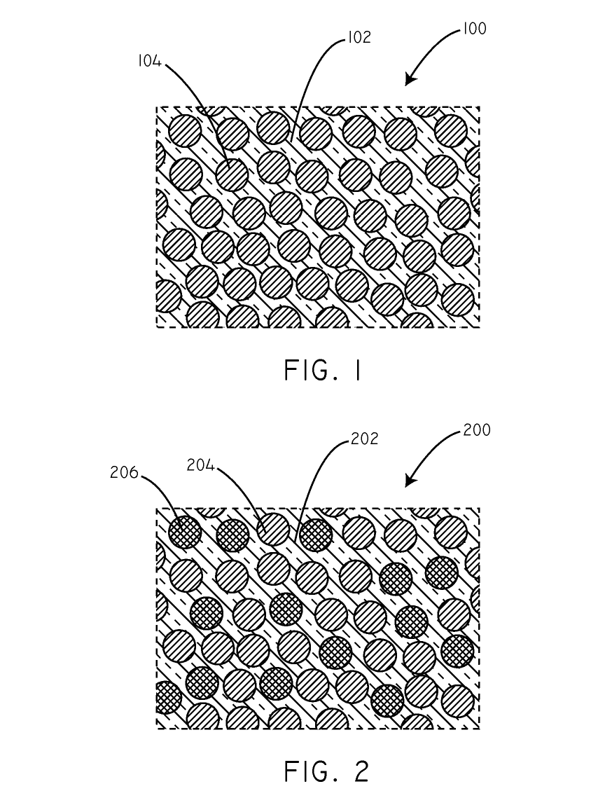

It will be appreciated that in some embodiments biodegradable composites can include more than one reinforcing metal 104. Referring now to FIG. 2, a schematic cross-sectional view of a degradable composite 200 including two different reinforcing metals 204, 206 is shown in accordance with at least one embodiment. In some embodiments, one of the degradable metals can be selected to have a longer degradation time than the other degradable metal.

In some embodiments devices can include more than one composite. Referring now to FIG. 3A, a schematic view is shown of a portion of a device 300 including a first biodegradable composite 302 and a second biodegradable composite 304. The first biodegradable composite 302 can have either the same or different properties as the second biodegradable composite 304. By way of example, the first biodegradable composite 302 can have either the same or different degradation rate as the second biodegradable composite 304. The first biodegradable composite 302 can have either the same or different structural properties as the second biodegradable composite 304.

It will be appreciated that the first and second composites can be selected so that properties of the device, such as structural properties, are effectively modulated to fit a desirable end use. By way of example, the first composite 302 can be selected to have a slower degradation rate than the second composite 304. The second composite 304 can be substantially surrounded by the first composite 302, and therefore protected from degradation until the first composite 302 is substantially eroded away. Therefore, the drop off in structural strength over time would be relatively small at first and then accelerate rapidly after the first composite 302 is eroded away.

Referring now to FIG. 3B, a schematic view is shown of a portion of a composite 350 in accordance with an embodiment of the invention. The composite 350 can include a degradable metal wire 358 coated with a first degradable polymer 360. The degradable metal wire 358 and the first degradable polymer 360 can be incorporated into a second biodegradable polymer matrix 356. In some embodiments, the first degradable polymer 360 can serve to reduce the degradation rate of the degradable metal wire 358. By way of example, the first degradable polymer 360 can be a hydrophobic polymer and can serve to prevent water from contacting the degradable metal wire 358. In other embodiments, the first degradable polymer 360 can serve to enhance the degradation rate of the degradable metal wire 358. For example, as described below, degradation of the first degradable polymer 360 can result in acidic break-down products that accelerate degradation of the degradable metal wire 358. The degradable metal wire(s) 358 and first degradable polymer 360 can take on the form of a mesh, woven scaffold, or a pre-impregnated substrate in some embodiments.

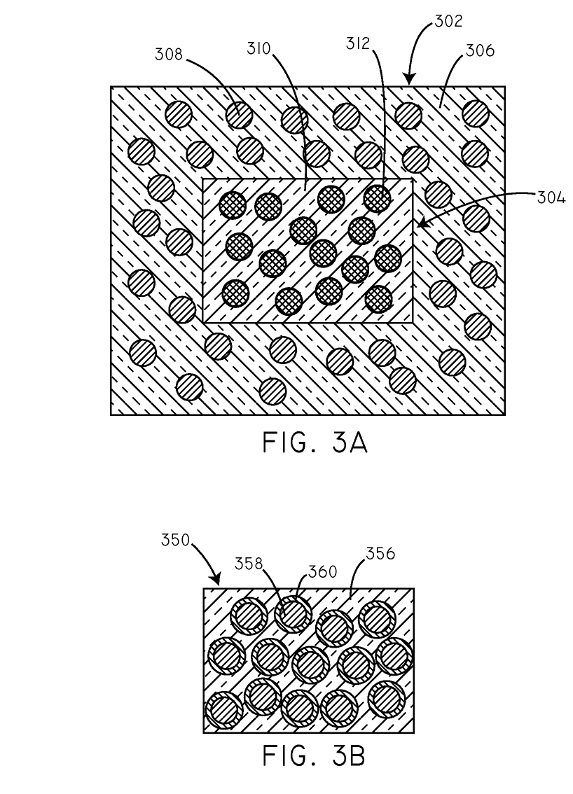

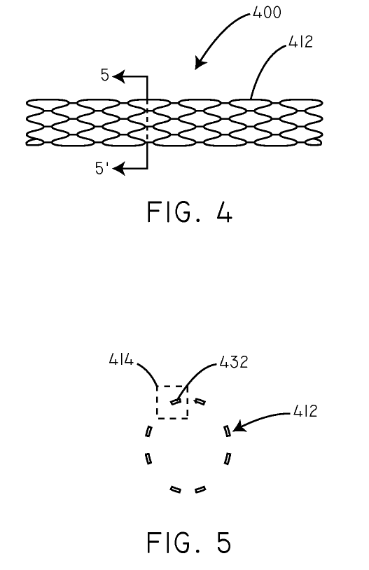

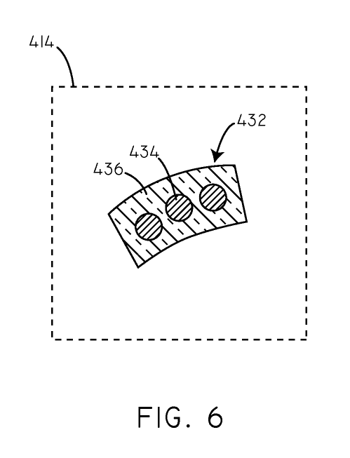

Composites includes herein can be used to form a variety of different implantable medical devices. Referring now to FIG. 4, a schematic view is shown of an exemplary implantable medical device. In FIG. 4, a stent 400 is shown in accordance with an embodiment herein. The stent 400 has a generally tubular shape and includes a plurality of struts 412. The stent 400 is configured to expand from a first diameter to a second diameter in order to engage a vessel wall when deployed in vivo. FIG. 5 is a cross-sectional view of the stent 400 as taken along lines 5-5′ of FIG. 4. The struts 412 can be made of a composite 432. FIG. 6 is an enlarged view of a portion 414 of FIG. 5. In this view, it can be seen that the composite 432 includes a polymeric matrix 436 and a reinforcing metal 434. The polymeric matrix 436 connects portions of the reinforcing metal 434 that are otherwise not connected at that discrete location.

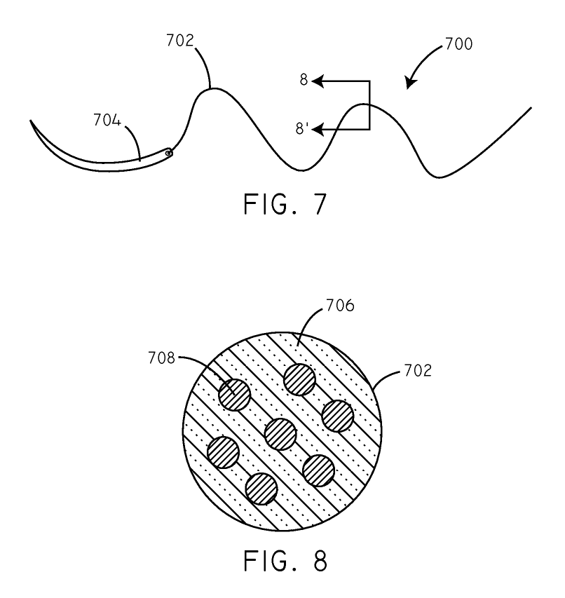

Referring now to FIG. 7, a schematic view is shown of another exemplary implantable medical device 700. In FIG. 7, a suturing system 700 is shown in accordance with an embodiment herein. The suturing system 700 includes a needle 704 attached to a suture string 702. FIG. 8 is a cross-sectional schematic view of the suture string 702 as taken along line 8-8′ of FIG. 7. The suture string 702 can be formed of a biodegradable composite including a polymeric matrix 706 and a reinforcing metal 708 as described herein.

### Degradable Polymers

Degradable polymers used with embodiments of the invention can include both natural or synthetic polymers. Examples of degradable polymers can include those with hydrolytically labile linkages in the polymeric backbone. Examples of degradable polymers can also include those with enzymatically labile linkages in the polymeric backbone. Degradable polymers of the invention can include both those with bulk erosion characteristics and those with surface erosion characteristics.

Synthetic degradable polymers can include, but are not limited to: degradable polyesters (such as poly(glycolic acid), poly(lactic acid), poly(lactic-co-glycolic acid), poly(dioxanone), polylactones (e.g., poly(caprolactone)), poly(β-hydroxybutyrate), poly(3-hydroxybutyrate), poly(3-hydroxyvalerate), poly(valerolactone), poly(tartronic acid), poly(B-malonic acid), polypropylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates and other carbonates such as trimethylene carbonate); degradable polyiminocarbonates; degradable polyarylates (such as tyrosine-based polyarylates); degradable polyorthoesters; degradable polyurethanes; degradable polyphosphazenes; and degradable polyhydroxyalkanoates; and copolymers thereof.

Natural or naturally-based degradable polymers can include polysaccharides and modified polysaccharides such as starch, cellulose, chitin, chitosan, and copolymers thereof.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)−]x[—O—(CH₂)₄—O—C(O)—C₆H₄—C(O)—]y,

where —C₆H₄—designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. n can be selected such that the molecular weight of the PEG block is between about 300 and about 4000. X and y can be selected so that the multiblock copolymer contains from about 55% up to about 80% PEG by weight. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure.

Degradable polyesteramides can include those formed from the monomers OH—x—OH, z, and COOH—y—COOH, wherein x is alkyl, y is alkyl, and z is leucine or phenylalanine.

Degradable polymeric materials can also be selected from: (a) non-peptide polyamino polymers; (b) polyiminocarbonates; (c) amino acid-derived polycarbonates and polyarylates; and (d) poly(alkylene oxide) polymers.

In an embodiment, the degradable polymeric material is composed of a non-peptide polyamino acid polymer. Exemplary non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, including two or three amino acid units having one of the following two structures illustrated below:

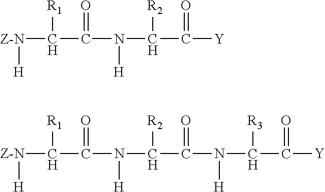

wherein the monomer units are joined via hydrolytically labile bonds at not less than one of the side groups R₁, R₂, and R₃, and where R₁, R₂, R₃ are the side chains of naturally occurring amino acids; Z is any desirable amine protecting group or hydrogen; and Y is any desirable carboxyl protecting group or hydroxyl. Each monomer unit comprises naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are degradable, and can provide zero-order release kinetics for the delivery of active agents in a variety of therapeutic applications. According to these aspects, the amino acids are selected from naturally occurring L-alpha amino acids, including alanine, valine, leucine, isoleucine, proline, serine, threonine, aspartic acid, glutamic acid, asparagine, glutamine, lysine, hydroxylysine, arginine, hydroxyproline, methionine, cysteine, cystine, phenylalanine, tyrosine, tryptophan, histidine, citrulline, ornithine, lanthionine, hypoglycin A, β-alanine, γ-amino butyric acid, α aminoadipic acid, canavanine, venkolic acid, thiolhistidine, ergothionine, dihydroxyphenylalanine, and other amino acids well recognized and characterized in protein chemistry.

Degradable polymers of the invention can also include polymerized polysaccharides such as those described in U.S. Publ. patent application No. 2005/0255142, entitled “COATINGS FOR MEDICAL ARTICLES INCLUDING NATURAL BIODEGRADABLE POLYSACCHARIDES”, U.S. Publ. patent application No. 2007/0065481, entitled “COATINGS INCLUDING NATURAL BIODEGRADABLE POLYSACCHARIDES AND USES THEREOF”, and in U.S. Application Ser. No. 60/782,957, entitled “HYDROPHOBIC DERIVATIVES OF NATURAL BIODEGRADABLE POLYSACCHARIDES”, all of which are herein incorporated by reference.

Degradable polymers of the invention can also include dextran based polymers such as those described in U.S. Pat. No. 6,303,148, entitled “PROCESS FOR THE PREPARATION OF A CONTROLLED RELEASE SYTEM”. Exemplary dextran based degradable polymers including those available commercially under the trade name OCTODEX.

Degradable polymers of the invention can further include collagen/hyaluronic acid polymers.

Degradable polymers of the invention can include multi-block copolymers, comprising at least two hydrolysable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolysable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione). The composition of the pre-polymers can be chosen in such a way that the maximum glass transition temperature of the resulting copolymer is below 37° C. at body conditions. To fulfill the requirement of a Tg below 37° C., some of the above-mentioned monomers or combinations of monomers can be more preferred than others. This may by itself lower the Tg, or the pre-polymer is initiated with a polyethylene glycol with sufficient molecular weight to lower the glass transition temperature of the copolymer. The degradable multi-block copolymers can include hydrolysable sequences being amorphous and the segments can be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained.

In some embodiments, the degradable polymer can be one for which degradation results in the formation of acidic degradation products. As just one example, degradation of polylactide results in the formation of lactic acid. While not intending to be bound by theory, it is believed that the formation of an acidic degradation product can enhance degradation of a reinforcing metal.

In some embodiments, a bioactive agent can also be incorporated into a polymeric matrix formed by the degradable polymer of the composite. Examples of bioactive agents can specifically include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, (poly)peptides, proteins, enzymes, extracellular matrix components, inhibitors, free radical scavengers, chelators, antioxidants, anti polymerases, antiviral agents, photodynamic therapy agents, and gene therapy agents.

### Reinforcing Degradable Metals

Embodiments of composites included herein can include a reinforcing degradable metal. The metal can be in various forms, by way of example the reinforcing metal can be in the form of a powder, granular mixture, fibers, rods, whiskers, fibrous woven, fibrous non-woven, spheres, or the like.

Exemplary reinforcing metals can include magnesium, iron, zinc, selenium, and oxides thereof, and alloys including at least one of the foregoing.

The relative proportion of the reinforcing metal in the composite can vary. In some embodiments the degradable composite can include at least about 5 wt. % of one or more reinforcing metals. In some embodiments the biodegradable composite can include at least about 10 wt. % of one or more reinforcing metals. In some embodiments the biodegradable composite can include at least about 20 wt. % of one or more reinforcing metals. In some embodiments the biodegradable composite can include at least about 40 wt. % of one or more reinforcing metals. In some embodiments the biodegradable composite can include from about 5 wt. % to about 90 wt. % of one or more reinforcing metals. In some embodiments the biodegradable composite can include from about 10 wt. % to about 40 wt. % of one or more reinforcing metals.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference. Nothing herein is to be construed as an admission that the inventors are not entitled to antedate any publication and/or patent, including any publication and/or patent cited herein.

The invention has been described with reference to various specific and preferred embodiments and techniques. However, it should be understood that many variations and modifications may be made while remaining within the spirit and scope of the invention.

### Further Embodiments

In an embodiment the invention includes a biodegradable implantable medical device. The device can include a biodegradable composite member comprising a polymeric matrix and a reinforcing metal disposed within the polymeric matrix, the biodegradable composite member configured to erode in vivo. In an embodiment, the metal is in elemental form. In an embodiment, the metal is an alloy. In an embodiment, the metal is selected from the group consisting of magnesium, zinc, selenium, and iron. In an embodiment, the metal comprises from about 5 wt. % to about 90 wt. % of the biodegradable composite member. In an embodiment, the metal comprises from about 10 wt. % to about 40 wt. % of the biodegradable composite member. In an embodiment, the metal comprises fibers. In an embodiment, the metal comprises a woven structure. In an embodiment, the metal comprising a particulate. In an embodiment, the polymeric matrix comprises a polymer that is hydrolytically labile and/or enzymatically labile. In an embodiment, degradation of the polymer results in an acidic degradation product. In an embodiment, the polymeric matrix comprising a hydrophilic polymer. In an embodiment, the polymeric matrix comprising polylactic acid. In an embodiment, the polymeric matrix comprising polylactide-co-glycolide. In an embodiment, the biodegradable composite member exhibits stiffness greater than an otherwise identical member including only the polymeric matrix. In an embodiment, the biodegradable composite member exhibits fracture resistance greater than an otherwise identical member including only the polymeric matrix. In an embodiment, the biodegradable composite member is configured to erode in vivo at least about 90% in mass over a period of time no longer than two years. In an embodiment, the biodegradable composite member comprises a tubular shape. In an embodiment, the biodegradable implantable device comprising a stent. In an embodiment, the biodegradable implantable medical device comprises a bioactive agent disposed within the polymeric matrix.

In an embodiment, the invention includes a method of making a biodegradable implantable device comprising contacting a polymer mixture with a reinforcing metal, the polymer mixture comprising a polymer that degrades under in vivo conditions and the reinforcing metal comprising a metal that produces substantially non-toxic erosion products. In an embodiment, the method includes contacting a polymer mixture with a reinforcing metal comprising injection molding the polymer mixture into contact with the reinforcing metal.

1. A biosorbable implantable medical device comprising:
a composite article comprising a polymeric matrix and a reinforcing metal disposed within the polymeric matrix, the composite article configured to erode in vivo.

2. The biosorbable implantable medical device of claim 1, wherein the metal is in elemental form.

3. The biosorbable implantable medical device of claim 1, wherein the metal is an alloy.

4. The biosorbable implantable medical device of claim 1, the metal selected from the group consisting of magnesium, zinc, selenium, and iron.

5. The biosorbable implantable medical device of claim 1, the metal comprising from about 5 wt. % to about 90 wt. % of the composite article.

6. The biosorbable implantable medical device of claim 1, the metal comprising from about 10 wt. % to about 40 wt. % of the composite article.

7. The biosorbable implantable medical device of claim 1, the metal comprising fibers.

8. The biosorbable implantable medical device of claim 1, the metal comprising a woven structure.

9. The biosorbable implantable medical device of claim 1, the metal comprising a particulate.

10. The biosorbable implantable medical device of claim 1, wherein the polymeric matrix comprises a polymer that is hydrolytically labile and/or enzymatically labile.

11. The biosorbable implantable medical device of claim 1, wherein degradation of the polymer results in an acidic degradation product.

12. The biosorbable implantable medical device of claim 1, the polymeric matrix comprising a hydrophilic polymer.

13. The biosorbable implantable medical device of claim 1, the polymeric matrix comprising polylactic acid.

14. The biosorbable implantable medical device of claim 1, the polymeric matrix comprising polylactide-co-glycolide.

15. The biosorbable implantable medical device of claim 1, wherein the composite article exhibits stiffness greater than an otherwise identical article including only the polymeric matrix.

16. The biosorbable implantable medical device of claim 1, wherein the composite article exhibits fracture resistance greater than an otherwise identical article including only the polymeric matrix.

17. The biosorbable implantable medical device of claim 1, the composite article configured to erode in vivo at least about 90% in mass over a period of time no longer than two years.

18. The biosorbable implantable medical device of claim 1, the composite article comprising a tubular shape.

19. The biosorbable implantable medical device of claim 1, the biosorbable implantable device comprising a stent.

20. The biosorbable implantable medical device of claim 1, further comprising a bioactive agent disposed within the polymeric matrix.

21. A method of making a biosorbable implantable device comprising contacting a polymer mixture with a reinforcing metal, the polymer mixture comprising a polymer that degrades under in vivo conditions and the reinforcing metal comprising a metal that produces substantially non-toxic erosion products.

22. The method of claim 21, wherein contacting a polymer mixture with a reinforcing metal comprising injection molding the polymer mixture into contact with the reinforcing metal.
